(19) United States
(12) Patent Application Publication
(10) Pub. No.: US 2018/0116745 A1
(43) Pub. Date: May 3, 2018

(54) MICROSCOPE DRAPE FOR STERILE ENVIRONMENTS

(71) Applicant: Premier Guard LLC, Houston, TX (US)

(72) Inventors: Charles Hubbs, Houston, TX (US); Shane Hubbs, Houston, TX (US)

(73) Assignee: Premier Guard LLC, Houston, TX (US)

(21) Appl. No.: 15/342,713

(22) Filed: Nov. 3, 2016

(51) Int. Cl. A61B 46/10; A61B 46/00; G02B 21/00

(52) U.S. Cl. CPC A61B 46/10; A61B 90/20; G02B 21/0012; A61B 46/40

(57) ABSTRACT

A drape for covering a microscope during a medical procedure includes a body portion, a tubular portion, and an elastic ring. The body portion is configured to drape over a body of the microscope. The tubular portion corresponds to an eyepiece tube of the microscope and is connected to the body portion at a first end and terminating at a second end. The elastic ring is positioned at the second end of the tubular portion. The elastic ring includes an elastic material and the elastic ring is sized to grip an eyepiece or the eyepiece tube of the microscope.

### TECHNICAL FIELD

The present disclosure relates to microscope drapes or covers used in sterile environments and more particularly relates to drapes or coverings for an eyepiece or eyepiece tube of a microscope for surgical or other sterilized purposes.

### BRIEF DESCRIPTION OF THE DRAWINGS

FIG. 1 is a perspective view of a cover draped over a surgical microscope assembly, according to one embodiment.

FIG. 2 is a plan view of an ocular portion of a cover with elastic rings, according to one embodiment.

FIG. 3 is a perspective side view of a cover positioned on a microscope, according to one embodiment.

FIG. 4 is a perspective view illustrating placement of an ocular portion of a cover on a microscope, according to one embodiment.

FIG. 5 is a perspective view illustrating an ocular portion of a cover after it has been positioned on a microscope, according to one embodiment.

FIG. 6 is a side view illustrating an elastic ring conforming to an eyepiece and/or eyepiece tube of a microscope, according to one embodiment.

FIG. 7 is a schematic flow chart diagram illustrating a method for infection control by covering a microscope for use during a medical procedure, according to one embodiment.

FIG. 8 is a plan view of a microscope drape or cover, according to one embodiment.

FIG. 9 is a plan view of another microscope drape or cover, according to one embodiment.

FIG. 10 is a plan view of a bonnet-style microscope drape or cover, according to one embodiment.

### DETAILED DESCRIPTION

Microscopes are often used for enlargement or viewing in surgical or operating environments. For example, surgical or operating microscopes may be used in a surgical setting to perform operations, such as microsurgery. Surgical microscopes may be used in fields of medicine such as dentistry, ear nose and throat (ENT) surgery, ophthalmic surgery, neurosurgery, or the like. It will be appreciated that the disclosure is not limited to any particular field of medicine or any particular field, but may apply to all fields of medicine or otherwise in which a microscope is used and required as part of a sterile environment.

It will be appreciated that the following disclosure uses surgical microscopes and environments as an example embodiment for sake of simplicity and understanding. However, it will be appreciated that the subject matter of the disclosure is equally applicable to any sterile environment in which microscopes are used, such as clean rooms in manufacturing plants or other sterilized environments.

Because surgical microscopes may be used in a surgery environment, contamination and sterility is extremely important to prevent cross-infection between patients. For example, the surgical microscope may be used in one surgery after another and it is important the microscope not be a source of contamination between patients. Infection control is one of the highest focus areas in a hospital to limit transferring contaminants from one patient to the next. To maintain sterility and prevent cross-contamination or cross-infection, the surgical microscope may be constructed of components that are easy to sterilize or disinfect. Additionally or alternatively, a drape or cover may be used to cover portions of the microscope and associated mechanisms so that those portions of the microscope remain sterile even if used during the surgery. The covers may be removed and replaced between surgeries and thus the time needed to disinfect the microscope may be significantly reduced. For example, only small portions of the microscope may need to be sterilized. However, drapes or covers for surgical microscopes may complicate usage of the microscope during surgery. For example, the drapes or covers may become displaced or tangled as they are manipulated by a medical worker, which may result in the need to re-drape the drape or cover during the procedure. Additionally, it may take some time to place the drape or cover over the surgical microscope.

Applicants have recognized that considerable benefit can be achieved by improving drapes or covers and how they are mounted or placed on surgical microscopes. For example, surgical microscopes include one or more eyepieces (i.e., oculars) through which a medical worker may look to view a surgical area or surgical equipment during a procedure. Thus, a drape or cover must generally allow a user to peer through the eyepiece during surgery and thus cannot completely cover a lens of the eyepiece. In order to secure portions of the drape or cover in a proper position around the eyepiece it may be necessary to use tape or a hook and loop fastener (such as Velcro®). In some cases, a drape may include perforated regions that can be torn out as holes corresponding to the eyepieces. However, the application of tape or hook and loop fastener can be time consuming and may not result in an ideally situated drape. Additionally, during use of the microscope, a user may grasp the oculars (eyepiece or eyepiece tube), which may cause the drape, hook and loop fastener, and/or tape to be moved from an optimal position. This may require re-draping or reapplying a fastener to get the drape or cover in the right position. Because surgeries are time sensitive, re-draping, re-taping, or resituating the drape or cover may cause unwanted delays, which delays result in increased costs associated with a surgery.

In light of the foregoing, Applicants have developed surgical drapes and covers and methods for mounting surgical drapes and covers. In one embodiment, a drape for covering a microscope during a medical procedure includes a body portion, a tubular portion, and an elastic ring. The body portion is configured to drape over a body of the microscope. The tubular portion corresponds to an eyepiece tube of the microscope and connects to the body portion at a first end and terminating at a second end. The elastic ring is positioned at the second end of the tubular portion. The elastic ring includes an elastic material and the elastic ring is sized to grip an eyepiece or the eyepiece tube of the microscope.

The cover and drape embodiments disclosed herein may provide improved functionality and sterility during procedures. In one embodiment, the elastic ring of a cover firmly grips the eyepiece and/or eyepiece tube to maintain sterility of the ocular portion of a microscope. The elastic ring is flexible so the ring can stretch to accommodate at least a slightly larger diameter. The cover may be applied simply by pushing or pulling the tubular portion, cover, or elastic ring over the oculars. Because the elastic ring is sized for the eyepiece or eyepiece tube of a specific microscope, the cover may or accommodate the oculars like a glove. The form fitting elastic ring may have a rubberized or gripping interior surface that keeps the cover in place significantly better than smooth plastic, even if the smooth plastic were to be secured using tape or a hook and loop fastener.

Because of the glove-like configuration of some embodiments, the time to put the drape on is faster and improves equipment functionality during procedures because adjustments to the equipment do not require the drape to be moved or fixed when the microscope is moved. During a surgical procedure doctors or medical personnel do not want to be bothered with the drape not fitting well or making an adjustment to the drape or cover. For example, the drape or cover may be used in delicate and intense surgical procedures, such as brain surgery, cardiovascular surgery, eye surgery, or the like. The drape or cover may be simply and quickly pulled onto the oculars to protect them from contamination, providing robust contamination control and time saving.

A detailed description of systems and methods consistent with embodiments of the present disclosure is provided below. While several embodiments are described, it should be understood that this disclosure is not limited to any one embodiment, but instead encompasses numerous alternatives, modifications, and equivalents. In addition, while numerous specific details are set forth in the following description in order to provide a thorough understanding of the embodiments disclosed herein, some embodiments may be practiced without some or all of these details. Moreover, for the purpose of clarity, certain technical material that is known in the related art has not been described in detail in order to avoid unnecessarily obscuring the disclosure.

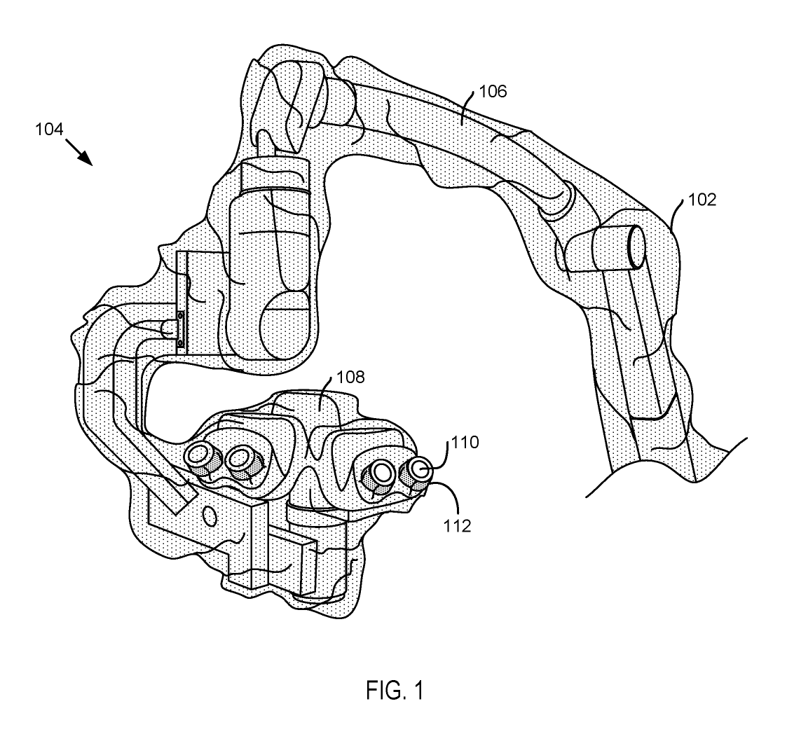

Turning to the figures, FIG. 1 is a perspective view showing a cover 102 draped over a surgical microscope assembly 104. The surgical microscope assembly includes an arm 106 supporting a microscope body 108. The microscope body 108 may include optical elements for magnifying objects or a region below the microscope body 108 and one or more eyepieces 110 for viewing the magnified object or region. For example, a user may look through an eyepiece 110 during a surgical procedure for assistance in seeing small objects. The cover 102 includes an arm portion covering the arm, a body portion covering the microscope body 108, and flexible rings 112 for at least partially covering an exterior of the eyepiece and/or an eyepiece tube. In the embodiment, of FIG. 1 the surgical microscope assembly 104 includes two ocular viewing regions for a total of four eyepieces 110 and eyepiece tubes. The cover 102 includes four flexible rings 112 to match the four eyepieces 112 or eyepiece tubes.

In one embodiment, the cover 102 is specially manufactured for a specific microscope assembly 104 or microscope body 104. For example, the cover 102 includes an arm portion corresponding to the arm 106, a body portion corresponding to a body of the microscope body 108, and flexible rings 112 corresponding to the four eyepieces 112 or eyepiece tubes. In one embodiment, the arm portion and the body portion are formed of a single plastic (e.g., polyethylene) sheet and the elastic ring 112 is formed from a different material such as polymer, rubber, latex, or the like. For example, the arm and body portion may be formed of a tube or flat sheet of plastic that has limited elasticity while the elastic ring 112 is formed of an at least partially elastic material which can stretch and then return to an original dimension or radius. The cover 102 may be formed disposable materials such that the cover 102 may be removed after a procedure and a new cover provided for a subsequent procedure. The body portion and/or the arm portion of the cover 102 may form a tube which may be spread over the arm 106 and/or microscope body 108 of the microscope assembly 104. In one embodiment, the body portion and/or the arm portion comprise a flat open sheet which can be draped and/or wrapped around the arm 106 and/or microscope body 108. For example, the sheet may be draped and wrapped around a portion of the microscope assembly 104 one or more times and secured with an elastic member, string, zip tie, or other elongated fastener to hold the sheet in place.

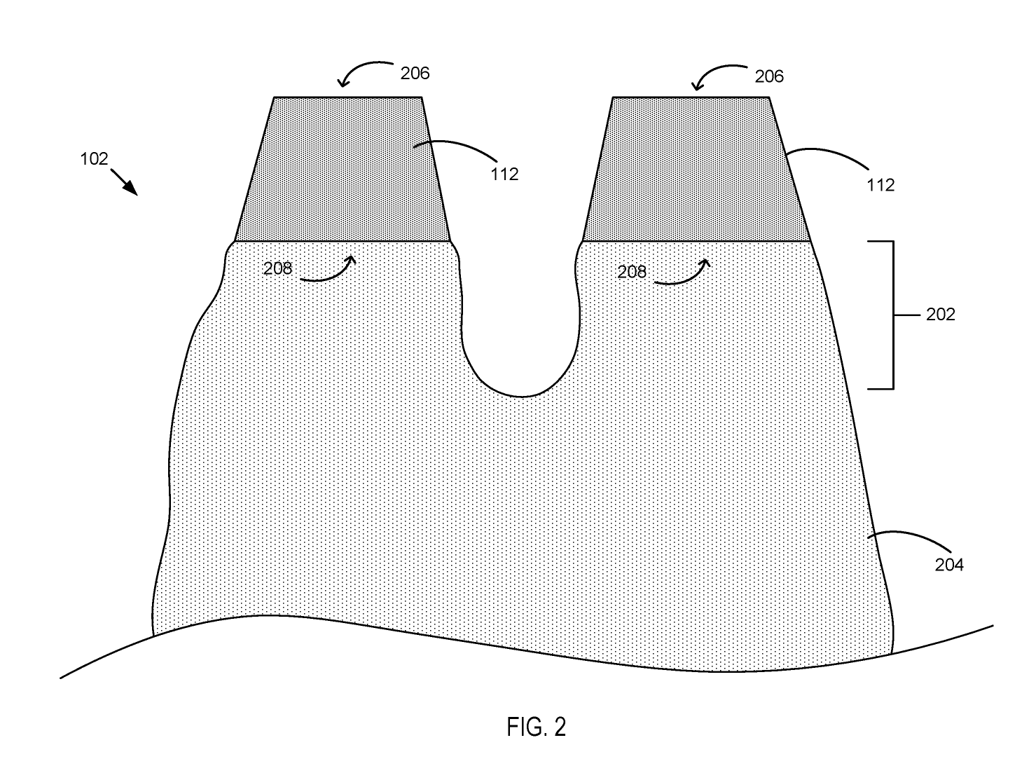

FIG. 2 is a side view illustrating an ocular portion 202 of a cover 102. The ocular portion of the cover 102 is shaped and sized to cover at least a portion of an eyepiece and/or an eyepiece tube of a cover 102. The ocular portion includes elastic rings 112, tubular portions 202 and a body portion 204.

The elastic rings 112 may correspond to two eyepieces or eyepiece tubes of a microscope. In one embodiment, multiple ocular portions may be included as part of a single cover. For example, a microscope may include more than one viewing port so that multiple medical workers can use the microscope at the same time. The elastic rings 112 include rings or tubes made from an elastic material. For example, the elastic rings 112 may be formed of a polymer that can stretch and return back to an original size. The elastic rings 112 are sized to encompass an eyepiece or eyepiece tube of a microscope. For example, the elastic rings 112 may have an inner diameter less than the eyepiece or eyepiece tube but large enough that the elastic rings can stretch to accommodate the eyepiece or eyepiece without reaching its elastic limit.

The elastic rings 112 may be flattened such that they have a length greater than a width. The elastic rings 112 may form a tube which allows an eyepiece or eyepiece tube to be positioned in the elastic rings. In one embodiment, the elastic rings 112 may comprise a first end 206 with an opening so that a user can look through an eyepiece and a second end 208 that connects to the tubular portions 202. The elastic rings 112 have an inside diameter that varies along a length of the elastic rings 112. For example, an inside surface of the elastic rings 112 may have a frusto conical shape with the first end 206 having a first diameter and the second end 208 having a second, larger diameter. The smaller first end 206 and larger second end 208 may allow the elastic rings 112 to be more easily slid or pulled onto an eyepiece or eyepiece tube while still having a small enough diameter to provide a firm grip on the eyepiece or eyepiece tube. In one embodiment, an elastic ring 112 may include a substantially constant diameter along a length of the elastic ring 112.

The elastic rings 112 may include a high friction material that can grip the eyepiece or eyepiece tube. For example, the elastic rings 112 may be formed of a rubber or have a rubberized surface that grips a material of an external of an eyepiece or eyepiece tube well. An interior surface of the elastic rings and/or a rubberized or other high-grip surface may directly contact the eyepiece tube. The gripping and size of the elastic rings 112 may cause the ocular portion to firmly hold the ocular portion in place. For example, the elastic rings 112 may remain in place even if a worker grabs or pushes on the ocular portion. This can significantly reduce the need for re-draping, resulting in the shortening of draping times and procedures.

The tubular portions 202 may have dimensions for accommodating an eyepiece tube. For example, the tubular portions 202 may have a length and diameter large enough to allow the ocular portion to be placed on the eyepieces and eyepiece tubes. The body portion 204 and the tubular portions 202 may be formed of the same material or same sheet of material. Additionally, the body portion 204 and the tubular portions 202 may also be the same material or same sheet of material as an arm portion of a cover.

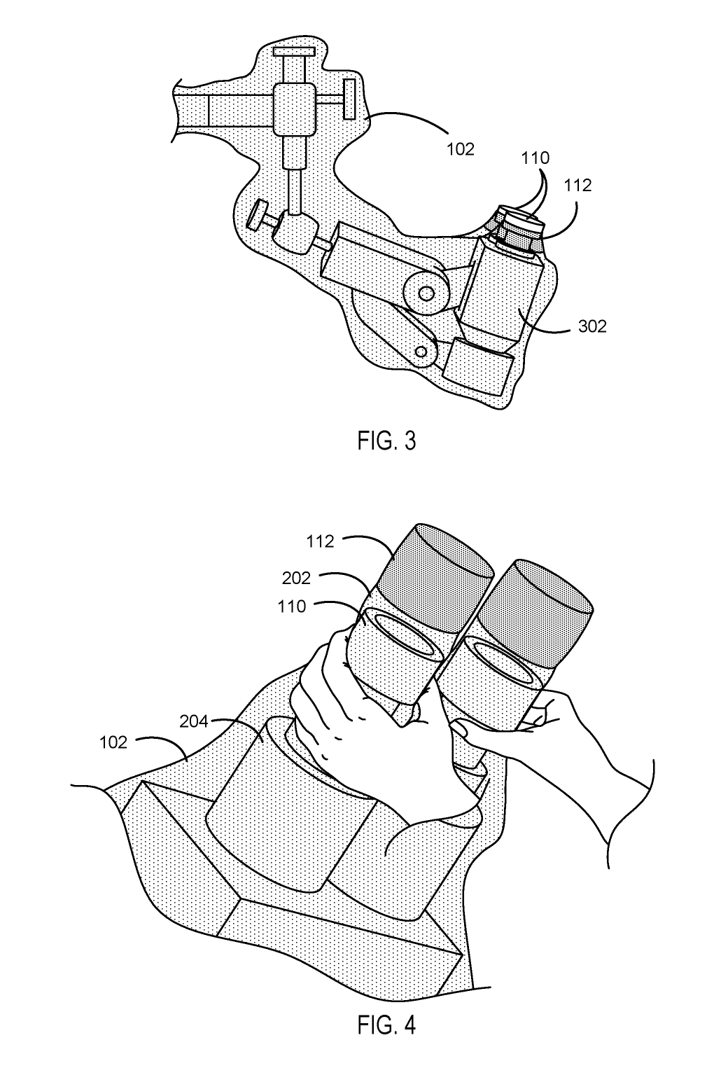

FIG. 3 illustrates a side view showing a cover 302 positioned on a microscope 302. The elastic rings 112 are shown positioned on an exterior of the eyepieces 110. The elastic rings 112 are positioned so that a user may peer through the eyepieces 110 to view a target operating area or operating tools. FIG. 4 is a perspective view illustrating an ocular portion of a cover 102 being placed on eyepieces of a microscope. The tubular portion 202 and the elastic rings 112 may be pulled downward over eyepiece tubes 204. As the tubular portion 202 and the elastic rings 112 are pulled downward, the elastic rings 112 may stretch to accommodate an outside diameter of the eyepiece 110 or eyepiece tube 204.

FIG. 5 illustrates a perspective view after an ocular portion of a cover 102 has been positioned on the eyepieces 110 and/or eyepiece tubes 204, according to one embodiment. The elastic rings 112 are positioned around the eyepieces 110 and eyepiece tubes 204 and provide a firm grip. The elastic rings 112 may include a gripping surface on an interior that, once positioned, limits movement of the elastic rings 112 and cover 102 with respect to the eyepieces 110 and eyepiece tubes 204. The elastic rings 112 may have stretched to accommodate the eyepieces 110 and eyepiece tubes 204 and, due to their elasticity, maintain a tight grip. A doctor or medical worker may be able to grab and/or manipulated the eyepieces 110 and/or eyepiece tubes 204 without causing the elastic rings 112 to be moved from a desired location on the eyepieces 110 and eyepiece tubes 204. Thus, there may be reduced need to adjust the cover 102 or re-drape a microscope during a procedure.

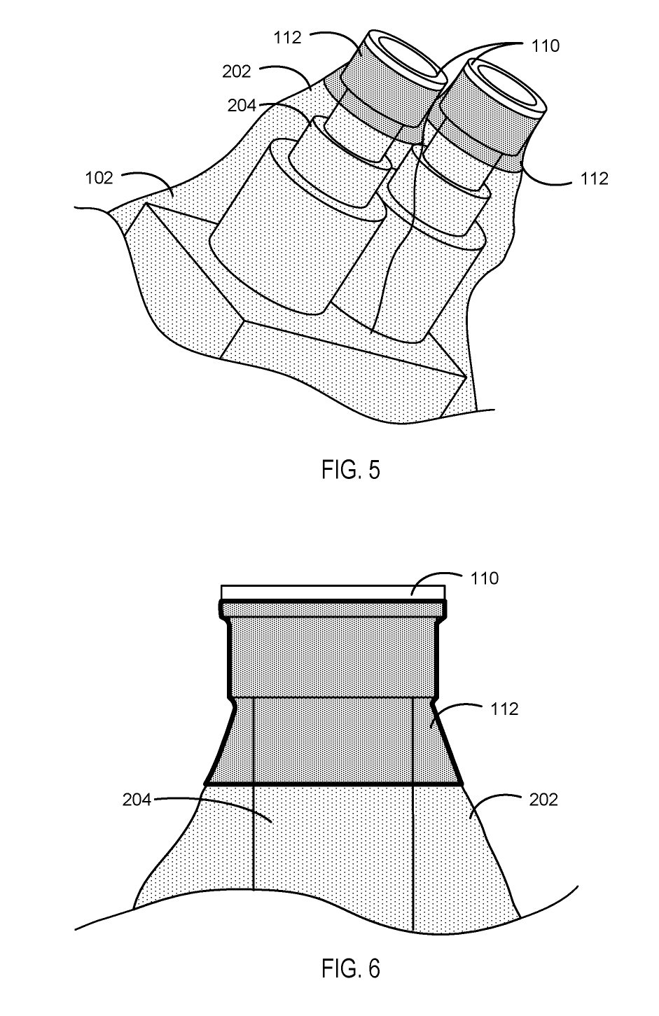

FIG. 6 illustrates a side view of an elastic ring 112 encompassing and gripping an eyepiece 110 and eyepiece tube 204, according to one embodiment. The elastic ring 112 includes a gripping interior surface. Due to its elasticity, the encompassing ring 112 stretches to accommodate and conform to an outside diameter of the eyepiece 110 and/or eyepiece tube 204. With the large amount of surface area in contact with the eyepiece 110 and/or eyepiece tube 204, the elastic ring 112 securely holds a cover and/or a tubular portion 202 of a cover in place.

The embodiments of elastic rings disclosed herein are given by way of example only. Considerable variation is possible without departing from the scope and teaching of the present disclosure. For example, the dimensions, including one or more of a thickness, length, material, or diameter (or variation in diameter) may vary significantly between embodiments. At least some embodiments expose an interior surface of the elastic ring to contact the eyepiece 110 and/or eyepiece tube 204 to provide secure gripping. For example, the tubular portion 202 or other portion of a cover may be formed of a plastic sheet or other material which does not provide grip, even if a force is applied to an exterior of the material using tape or a hook and loop fastener. In at least one embodiment, the rubberized or other gripping surface of the elastic ring 112 provides greater friction and also conforms to an outside diameter of the eyepiece 110 and/or eyepiece tube 204 to provide secure gripping. Additionally, the elastic ring allows for increased ease in positioning a cover on a microscope because not additional fasteners (such as tape or a hook and loop fastener) is required.

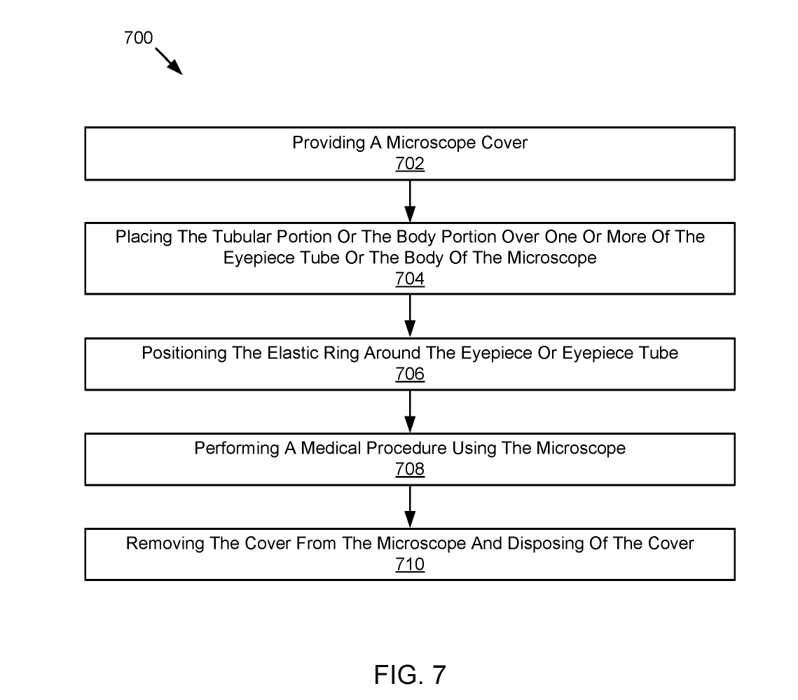

FIG. 7 is a schematic flow chart diagram illustrating an example method 700 for infection control by covering a microscope for use during a medical procedure. The method 700 may be performed by a medical worker or technician in preparation for, during, or after a medical procedure.

The method 700 includes providing 702 a microscope cover. The microscope cover may include one or more of a tubular portion, a body portion, and an elastic ring. The tubular portion or the body portion is for covering an eyepiece tube or body of the microscope. The elastic ring is connected to the tubular portion or the body portion. The elastic ring includes an elastic material and is sized to grip an eyepiece or the eyepiece tube of the microscope. The method 700 includes placing 704 the tubular portion or the body portion over one or more of the eyepiece tube or the eyepiece of the microscope. The method 700 includes positioning 706 the elastic ring around the eyepiece or eyepiece tube. Positioning 706 the elastic ring around the eyepiece or eyepiece tube may include: sliding the tubular portion or the elastic ring down over the eyepiece tube; and/or stretching the elastic ring to expand and grip the eyepiece tube or eyepiece. The method 700 may further include performing 708 a medical procedure using the microscope. During the medial procedure a user may look through or capturing images through the eyepiece. The method 700 further includes removing 710 the cover from the microscope and disposing of the cover. In one embodiment, the method 700 may be repeated for a second procedure by providing a second cover, positioning the second cover over the microscope, and performing a second medical procedure using the microscope in a similar manner.

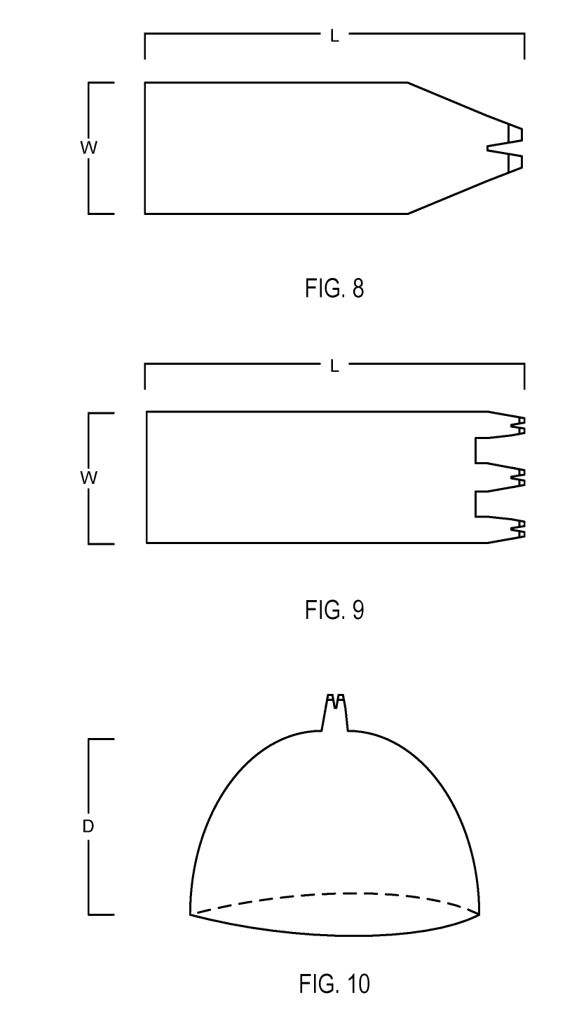

FIGS. 8-10 illustrate embodiments of microscope covers. Because doctors and medical professionals will often use microscopes with a cover, proper fitting of a cover can be important for not only the sale of the cover, but also for the microscope itself. Thus proper dimensions and high quality disposable covers that are convenient and easy to use are desirable. FIG. 8 is a plan view of a microscope drape or cover. The drape or cover includes two elastic rings to cover a pair of eyepieces or eyepiece tubes. The microscope cover may be sized to fit a specific microscope or the ocular portion of a specific microscope. By way of example, a microscope drape head cover for a Ziess OPMI® may have dimensions of (width×length) 20 in×30 in or 20 in×65 in and the elastic rings may be sized to fit a 48 mm lens.

FIG. 9 is a plan view of another microscope drape or cover. The drape or cover includes six elastic rings, two for each ocular portion. The microscope cover may be sized to fit a specific microscope or the ocular portion of a specific microscope. By way of example, a microscope drape for a Ziess® MD may have dimensions of (width×length) 46 in×64 in, 46 in×80 in, 46 in×105 in, or the like and the elastic rings may be sized to fit a 65 mm lens.

FIG. 10 is a plan view of a bonnet style microscope drape or cover. The drape or cover includes two elastic rings to cover a pair of eyepieces or eyepiece tubes. The microscope cover may be sized to fit a specific microscope or the ocular portion of a specific microscope. By way of example, a microscope drape head cover for a Ziess OPMI® may have a depth of 22 in.

### Examples

The following examples pertain to further embodiments.

Example 1 is a drape or cover for covering a microscope during a medical procedure that includes a body portion, a tubular portion, and an elastic ring. The body portion is configured to drape over a body and/or arm of the microscope. The tubular portion corresponds to an eyepiece tube of the microscope. The tubular portion is connected to the body portion at a first end and terminates at a second end. An elastic ring is positioned at the second end of the tubular portion. The elastic ring includes an elastic material and is sized to grip an eyepiece or the eyepiece tube of the microscope.

In Example 2, one or more of the tubular portion or body portion as in Example 1 includes a first material and the elastic ring includes a second material, wherein the elastic ring is bonded to the tubular portion.

In Example 3, at least a portion of the elastic ring as in any of Examples 1-2 includes a diameter smaller than a diameter of the eyepiece or the eyepiece tube. The elastic ring is configured to grip the eyepiece or eyepiece tube to limit movement of the elastic ring with respect to the eyepiece or eyepiece tube when the elastic ring is positioned around the eyepiece or eyepiece tube.

In Example 4, the elastic ring as in any of Examples 1-3 includes a width and a height greater than the width. The width includes a distance between an interior and exterior of the elastic ring and the height includes a dimension parallel to a line normal to an opening of the elastic ring.

In Example 5, an interior surface of the elastic ring as in any of Examples 1-4 forms a frusto conical shape or passage through the elastic ring.

In Example 6, the elastic ring as in any of Examples 1-5 includes a first end and a second end, wherein the first end of the elastic ring is attached to the second end of the tubular portion. The second end of the elastic ring includes an opening to allow a user to look through a lens or eyepiece of the microscope.

In Example 7, the first end of the elastic ring as in Example 6 includes a larger opening size than the second end of the elastic ring.

In Example 8, when the drape as in any of Examples 1-6 is positioned on the microscope, the body portion covers a body portion of the microscope, the tubular portion covers a portion of an eyepiece tube of the microscope, and the elastic ring covers and grips at least a portion of the eyepiece tube or an eyepiece of the microscope.

In Example 9, the second end of the tubular portion as in any of Examples 1-8 terminates with an opening and wherein the elastic ring forms an opening, wherein when mounted on the microscope the opening of the tubular portion and the opening of the elastic ring allows a user to look through the eyepiece to use the microscope.

In Example 10, the body portion as in any of Examples 1-9 includes a length greater than a width to cover the body and an arm on which the microscope is mounted.

In Example 11, the tubular portion as in any of Examples 1-10 includes a first tubular portion, the eyepiece tube of the microscope includes a first eyepiece tube, the eyepiece includes a first eyepiece, and the elastic ring includes a first elastic ring. The drape further includes a second tubular portion and a second elastic ring. The second tubular portion corresponds to a second eyepiece tube of the microscope. The second elastic ring encircles an end of the second tubular portion. The second elastic ring includes an elastic material and the second elastic ring is sized to grip a second eyepiece or the second eyepiece tube of the microscope.

Example 12 is a drape or cover for covering at least a portion of a microscope during a medical procedure. The cover includes a tubular portion or a body portion for covering at least a portion of a body or an eyepiece tube of the microscope. The cover also includes an elastic ring connected to the tubular portion or the body portion. The elastic ring includes an elastic material and the elastic ring is sized to grip an eyepiece or the eyepiece tube of the microscope.

In Example 13, the tubular portion or the body portion as in Example 12 includes a first material and the elastic ring includes a second material. The elastic ring is bonded to the tubular portion or the body portion.

In Example 14, at least a portion of the elastic ring in any of Examples 12-13 includes an inside diameter smaller than an outside diameter of the eyepiece or the eyepiece tube. The elastic ring is configured to grip the eyepiece or eyepiece tube to limit movement of the elastic ring with respect to the eyepiece or eyepiece tube when the elastic ring is positioned around the eyepiece or eyepiece tube.

In Example 15, the elastic ring as in any of Examples 12-14 includes a width and a height greater than the width. The width includes a distance between an interior and exterior of the elastic ring and the height includes a dimension parallel to a line normal to an opening of the elastic ring.

In Example 16, an interior surface of the elastic ring in any of Examples 12-15 forms a frusto conical shape or passage through the elastic ring.

In Example 17, the elastic ring as in any of Examples 12-16 includes a first end and a second end, wherein the first end of the elastic ring is attached to the tubular portion or the body portion and wherein the second end of the elastic ring comprises an opening.

In Example 18, the first end of the elastic ring as in Example 17 includes a larger opening size than the second end of the elastic ring.

In Example 19, when the cover as in any of Examples 12-18 is positioned on the microscope the tubular portion or the body portion covers a body portion or an eyepiece tube of the microscope and the elastic ring covers and grips at least a portion of the eyepiece tube or the eyepiece of the microscope.

In Example 20, when mounted on the microscope the opening of the elastic ring allows a user to look through the eyepiece to use the microscope.

In Example 21, the eyepiece tube of the microscope as in any of Examples 12-20 includes a first eyepiece tube, the eyepiece includes a first eyepiece, and the elastic ring includes a first elastic ring. The drape further includes a second elastic ring connected to the tubular portion or the body portion, wherein the second elastic ring comprises an elastic material and wherein the second elastic ring is sized to grip a second eyepiece or the second eyepiece tube of the microscope.

Example 22 is a method for covering a microscope for use during a medical procedure. The method includes providing a microscope cover that includes a tubular portion or body portion and an elastic ring. The tubular portion or a body portion is for covering an eyepiece tube or body of the microscope. The elastic ring is connected to the tubular portion or the body portion. The elastic ring includes an elastic material and the elastic ring is sized to grip an eyepiece or the eyepiece tube of the microscope. The method includes placing the tubular portion or the body portion over one or more of the eyepiece tube or the eyepiece of the microscope. The method further includes positioning the elastic ring around the eyepiece or eyepiece tube. The elastic ring is positioned around the eyepiece or eyepiece tube by one or more of sliding the tubular portion or the elastic ring down over the eyepiece tube or stretching the elastic ring to expand and grip the eyepiece tube or eyepiece.

In Example 23, the method of Example 22 further includes performing a medical procedure using the microscope, wherein using the microscope comprises looking through or capturing images through the eyepiece.

In Example 24, the method as in any of Examples 22-23 further includes removing the cover from microscope and disposing of the cover.

In Example 25, the microscope cover as in any of Examples 22-24 includes a first microscope cover and the medical procedure includes a first medical procedure. The method further includes providing a second cover, positioning the second cover over the microscope, and performing a second medical procedure using the microscope.

Reference throughout this specification to “an example” means that a particular feature, structure, or characteristic described in connection with the example is included in at least one embodiment of the present disclosure. Thus, appearances of the phrase “in an example” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on its presentation in a common group without indications to the contrary. In addition, various embodiments and examples of the present disclosure may be referred to herein along with alternatives for the various components thereof. It is understood that such embodiments, examples, and alternatives are not to be construed as de facto equivalents of one another, but are to be considered as separate and autonomous representations of the present disclosure.

Although the foregoing has been described in some detail for purposes of clarity, it will be apparent that certain changes and modifications may be made without departing from the principles thereof. It should be noted that there are many alternative ways of implementing both the processes and apparatuses described herein. Accordingly, the present embodiments are to be considered illustrative and not restrictive.

Those having skill in the art will appreciate that many changes may be made to the details of the above-described embodiments without departing from the underlying principles of the disclosure. The scope of the present disclosure should, therefore, be determined only by the following claims.

What is claimed is:

1. A drape for covering a microscope during a medical procedure, the drape comprising:
a body portion configured to drape over a body of the microscope;
a tubular portion corresponding to an eyepiece tube of the microscope, the tubular portion connecting to the body portion at a first end and terminating at a second end; and
an elastic ring positioned at the second end of the tubular portion, wherein the elastic ring comprises an elastic material and wherein the elastic ring is sized to grip an eyepiece or the eyepiece tube of the microscope.

2. The drape of claim 1, wherein one or more of the tubular portion or body portion comprises a first material and the elastic ring comprises a second material, wherein the elastic ring is bonded to the tubular portion.

3. The drape of claim 1, wherein at least a portion of the elastic ring comprises a diameter smaller than a diameter of the eyepiece or the eyepiece tube, wherein the elastic ring is configured to grip the eyepiece or eyepiece tube to limit movement of the elastic ring with respect to the eyepiece or eyepiece tube when the elastic ring is positioned around the eyepiece or eyepiece tube.

4. The drape of claim 1, wherein the elastic ring comprise a width and a height greater than the width, wherein the width comprises a distance between an interior and exterior of the elastic ring and the height comprises a dimension parallel to a line normal to an opening of the elastic ring.

5. The drape of claim 4, wherein an interior surface of the elastic ring forms a frusto conical shape or passage through the elastic ring.

6. The drape of claim 1, wherein the elastic ring comprises a first end and a second end, wherein the first end of the elastic ring is attached to the second end of the tubular portion and wherein the second end of the elastic ring comprises an opening.

7. The drape of claim 6, wherein the first end of the elastic ring comprises a larger opening size than the second end of the elastic ring.

8. The drape of claim 1 wherein when the drape is positioned on the microscope the body portion covers a body portion of the microscope, the tubular portion covers a portion of an eyepiece tube of the microscope, and the elastic ring covers and grips at least a portion of the eyepiece tube or an eyepiece of the microscope.

9. The drape of claim 1, wherein the second end of the tubular portion terminates with an opening and wherein the elastic ring forms an opening, wherein when mounted on the microscope the opening of the tubular portion and the opening of the elastic ring allows a user to look through the eyepiece to use the microscope.

10. The drape of claim 1, wherein the body portion comprises a length greater than a width to cover the body and an arm on which the microscope is mounted.

11. The drape of claim 1, wherein the tubular portion comprises a first tubular portion, the eyepiece tube of the microscope comprises a first eyepiece tube, the eyepiece comprises a first eyepiece, and the elastic ring comprises a first elastic ring, the drape further comprising:
a second tubular portion corresponding to a second eyepiece tube of the microscope; and
a second elastic ring encircling an end of the second tubular portion, wherein the second elastic ring comprises an elastic material and wherein the second elastic ring is sized to grip a second eyepiece or the second eyepiece tube of the microscope.

12. A cover for covering at least a portion of a microscope during a medical procedure, the cover comprising:
a tubular portion or a body portion for covering at least a portion of a body or an eyepiece tube of the microscope; and
an elastic ring connected to the tubular portion or the body portion, wherein the elastic ring comprises an elastic material and wherein the elastic ring is sized to grip an eyepiece or the eyepiece tube of the microscope.

13. The cover of claim 12, wherein the tubular portion or the body portion comprises a first material and the elastic ring comprises a second material, wherein the elastic ring is bonded to the tubular portion or the body portion.

14. The cover of claim 12, wherein at least a portion of the elastic ring comprises an inside diameter smaller than an outside diameter of the eyepiece or the eyepiece tube, wherein the elastic ring is configured to grip the eyepiece or eyepiece tube to limit movement of the elastic ring with respect to the eyepiece or eyepiece tube when the elastic ring is positioned around the eyepiece or eyepiece tube.

15. The cover of claim 12, wherein the elastic ring comprise a width and a height greater than the width, wherein the width comprises a distance between an interior and exterior of the elastic ring and the height comprises a dimension parallel to a line normal to an opening of the elastic ring.

16. The cover of claim 15, wherein an interior surface of the elastic ring forms a frusto conical shape or passage through the elastic ring.

17. The cover of claim 12, wherein the elastic ring comprises a first end and a second end, wherein the first end of the elastic ring is attached to the tubular portion or the body portion and wherein the second end of the elastic ring comprises an opening.

18. The cover of claim 17, wherein the first end of the elastic ring comprises a larger opening size than the second end of the elastic ring.

19. The cover of claim 12, wherein when the cover is positioned on the microscope the tubular portion or the body portion covers a body portion or an eyepiece tube of the microscope and the elastic ring covers and grips at least a portion of the eyepiece tube or the eyepiece of the microscope.

20. The cover of claim 12, wherein when mounted on the microscope the opening of the elastic ring allows a user to look through the eyepiece to use the microscope.

21. The cover of claim 12, wherein the eyepiece tube of the microscope comprises a first eyepiece tube, the eyepiece comprises a first eyepiece, and the elastic ring comprises a first elastic ring, the cover further comprising:
a second elastic ring connected to the tubular portion or the body portion, wherein the second elastic ring comprises an elastic material and wherein the second elastic ring is sized to grip a second eyepiece or the second eyepiece tube of the microscope.

22. A method for covering a microscope for use during a medical procedure, the method comprising:
providing a microscope cover comprising,
a tubular portion or a body portion for covering an eyepiece tube or body of the microscope; and
an elastic ring connected to the tubular portion or the body portion, wherein the elastic ring comprises an elastic material and wherein the elastic ring is sized to grip an eyepiece or the eyepiece tube of the microscope;
placing the tubular portion or the body portion over one or more of the eyepiece tube or the eyepiece of the microscope; and
positioning the elastic ring around the eyepiece or eyepiece tube by one or more of
sliding the tubular portion or the elastic ring down over the eyepiece tube, or
stretching the elastic ring to expand and grip the eyepiece tube or eyepiece.

23. The method of claim 22, further comprising performing a medical procedure using the microscope, wherein using the microscope comprises looking through or capturing images through the eyepiece.

24. The method of claim 23, further comprising removing the cover from microscope and disposing of the cover.

25. The method of claim 24, wherein the microscope cover comprises a first microscope cover and the medical procedure comprises a first medical procedure, the method further comprising providing a second cover, positioning the second cover over the microscope, and performing a second medical procedure using the microscope.
